(19) United States
(12) Patent Application Publication
(10) Pub. No.: US 2004/0161845 A1
(43) Pub. Date: Aug. 19, 2004

(54) Heating apparatus

(76) Inventors: Ramon E. Poo, Miami, FL (US); Camillo Ricordi, Miami, FL (US)

Correspondence Address: Gregory A. Nelson, Esq., Akerman, Senterfitt & Eidson, P.A., 222 Lakeview Avenue, Suite 400, P.O. Box 3188, West Palm Beach, FL, 33402-3188, US

(21) Appl. No.: 10/366,965

(22) Filed: Feb. 14, 2003

(51) Int. Cl. C12N 5/02; C12M 3/08

(52) U.S. Cl. 435/381; 435/308.1

(57) ABSTRACT

A system for extracting cells from organs includes a digestion chamber for containing a physiologically compatible medium with at least one protease. The chamber has at least one inlet and at least one outlet, and retains the organ and permits cells to exit the outlet. A conduit is provided for supplying the physiologically compatible medium to the digestion chamber. A heating apparatus has a heating surface and structure for removably securing an adjustable length of the conduit against the heating surface of the heating apparatus. The conduit is removably secured against the heating surface to heat the physiologically compatible medium. A method for extracting cells from organs and a heating apparatus are also disclosed.

### FIELD OF THE INVENTION

This invention relates generally to heating apparatus, and more particularly to heating apparatus for liquids.

### BACKGROUND OF THE INVENTION

Scientists are currently researching possible applications for isolating cells from parent organs, such as the liver, spleen, kidney, adrenal, and pancreas. Some research that has been conducted on the clinical application of isolated cells has involved groups of cells called the Islets of Langerhans that have been isolated from the pancreas. An application for the Islet of Langerhans cells is as a treatment for diabetic patients. Patients with diabetes have Islets of Langerhans that do not function properly, and therefore, do not produce enough insulin. Some clinical research is aimed at developing a procedure for transplanting functioning Islets of Langerhans into diabetic patients to restore the insulin producing ability of the patient. Clinical research of such treatments requires isolated Islets of Langerhans cells, but these cells must be isolated while still viable. Viable isolated cells are mostly obtained from organs of the very recently deceased. The apparatus and method for isolating the cells should be able to extract isolated cells with as little damage to the cells as possible.

Many different methods and approaches have been attempted to isolate viable cells from their respective parent organs. Prior methods, however, have produced isolated cells with some cell destruction. This cell destruction can result from the relatively severe mechanical stimulation that is used to isolate these cells from an organ.

One method that attempts to overcome the loss of damaged cells due to relatively severe mechanical stress is described in U.S. Pat. No. 5,079,160, to Lacy, et al, the disclosure of which is incorporated fully by reference. The method disclosed by Lacy, et al. comprises the steps of: placing an organ or a piece of an organ in a digestion chamber along with marble agitators; distending the organ or a piece of the organ with physiologically compatible medium containing a protease; continuously recirculating that medium; and separating the isolated cells. Lacy, et al. also described the application of heat to increase the temperature of the medium to activate the protease. Where the protease is collagenase, the heating should increase the temperature of the medium containing the protease to between about 28-38° C., and preferably about 37° C.

Any manner of heating the medium containing the protease is acceptable so long as the protease is not damaged. In practical applications in the laboratory, however, cellular extraction is performed with beakers and other containers, and the cellular extraction itself is performed in a chamber which retains the organ for contacting with the medium and protease, and permits the exit of extracted cells. Flexible laboratory tubing routes the medium between stages of the process.

The heating of the medium in one aspect occurs in a heating coil that is immersed in a hot water bath. Inlet tubing carries the medium to the heating coil, and separate outlet tubing carries the heated medium from the heating coil. The heating assembly therefore requires both inlet and outlet tubing, as well as a heating coil, which all must be assembled prior to operation and then disassembled, cleaned and sterilized prior to the next use. The assembly, cleaning and sterilization steps are time consuming and add to the expense of cellular extraction.

### SUMMARY OF THE INVENTION

A heating apparatus for cell extraction has a heating surface and structure for removably securing a length of flexible conduit against the heating surface of the heating apparatus. This structure can be a cover. The heating apparatus preferably has a heating surface that is arcuate or substantially cylindrical. The conduit supplying the physiologically compatible medium to the digestion chamber can be wrapped about the heating apparatus so as to contact the heating surface. The heating apparatus can have structure for controlling the amount of heat transferred by the heating surface to the flexible conduit.

A system for extracting cells from organs includes a digestion chamber for containing a physiologically compatible medium with at least one protease. The chamber has at least one inlet and at least one outlet, and preferably also a segregation means for retaining the organ and permitting cells to exit the outlet. A flexible conduit is provided for supplying the physiologically compatible medium to the digestion chamber. A heating apparatus has a heating surface and structure for removably securing a length of the flexible conduit against the heating surface.

A method for extracting cells from organs includes the steps of providing a digestion chamber containing a physiologically compatible medium with at least one protease. The chamber has at least one inlet and at least one outlet, and preferably also a segregation means for retaining the organ and permitting cells to exit the outlet. At least one conduit is provided for supplying the physiologically compatible medium to the digestion chamber. A heating apparatus is provided having a heating surface and structure for removably securing an adjustable length of the conduit against the heating surface. The conduit is positioned against the heating surface such that physiologically compatible medium flowing through the conduit will be heated. The amount of heat that is supplied by the heating surface to the conduit can be adjusted to control the heating of the fluid flowing through the conduit. Alternatively or additionally, the length of conduit in contact with the heating surface can be adjusted to control the temperature of the medium entering the digestion chamber.

The heater of the invention can be provided as a stand-alone unit or combined with other apparatus for cell extraction. In one embodiment, the heater is combined with a cell extraction assembly which includes a digestion chamber and structure for shaking the digestion chamber to facilitate cell extraction.

### BRIEF DESCRIPTION OF THE DRAWINGS

There are shown in the drawings embodiments which are presently preferred, it being understood, however, that the invention is not limited to the precise arrangements and instrumentalities shown, wherein:

FIG. 1 is a schematic diagram illustrating a system and method for extracting cells from organs.

FIG. 2 is a perspective view, partially in phantom, of a heater apparatus according to the invention.

FIG. 3 is a perspective view of an apparatus for extracting cells.

FIG. 4 is a perspective view of a heating apparatus according to the invention without a cover.

FIG. 5 is a perspective view of a heating apparatus according to the invention with a cover.

### DETAILED DESCRIPTION OF THE PREFERRED EMBODIMENTS

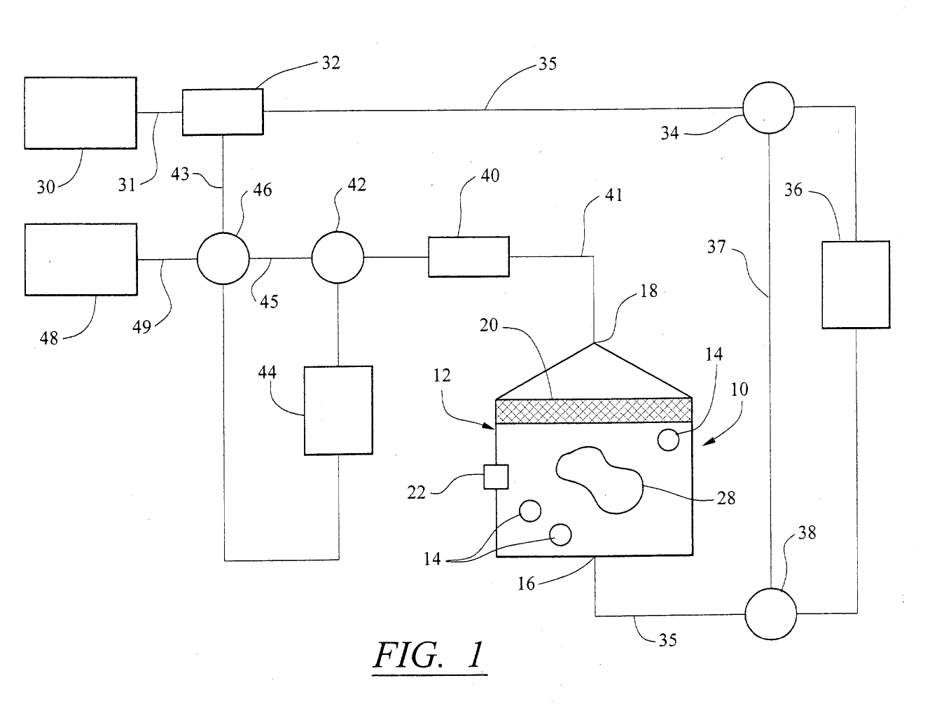

An example of a system and method for isolating cells from organs is shown in FIG. 1. In FIG. 1, the apparatus 10 is shown with the digestion chamber 12 having inlet 16 and outlet 18. The digestion chamber 12 can be connected to a recirculating system as shown in FIG. 1; however, the invention is not limited to a recirculating system as a non-recirculating system can be used. An organ 28, in whole or in part, is positioned in the digestion chamber 12 and a physiologically compatible medium with at least one protease is caused to flow through the digestion chamber 12. The physiologically compatible medium can flow through the inlet 16, the digestion chamber 12, and exit through the outlet 18. The digestion chamber 12 can include a segregation means 20 that permits the physiologically compatible medium and the isolated cells to exit the digestion chamber 12 while retaining the organ 28 inside the digestion chamber 12. At least one agitation member 14 can be provided in the digestion chamber 12. The agitation member 14 is preferably substantially spherical and is moved within the digestion chamber to agitate the organ 28 and facilitate the release of the cells. In one embodiment, the agitation members 14 are moved by gently shaking the digestion chamber 12. Finally, the isolated cells are collected.

The physiologically compatible medium can be heated prior to entering the digestion chamber 12. Before the physiologically compatible medium is introduced to the digestion chamber 12, a pump 32 can pump the physiologically compatible medium through a conduit 35 to a heating apparatus 36. The heating apparatus 36 heats the physiologically compatible medium to a temperature selected to maximize the effect of the protease. As is known in the art, the catalytic rate of a protease can be improved within a range of temperatures; therefore, the temperature selected to heat the physiologically compatible medium can be dependent on a variety of factors, including the specific protease or combination of proteases used. Accordingly, physiologically compatible medium can be heated to a temperature between 28° C.-38° C. For example, the physiologically compatible medium can be heated to about 37° C.; however, the invention is not limited in this regard, as a combination of factors are used to determine the temperature to which to heat the physiologically compatible medium.

After the physiologically compatible medium is heated to the proper temperature, physiologically compatible medium can be flowed through conduit 35 into the digestion chamber 12 through the inlet 16. The physiologically compatible medium with at least one protease can isolate the cells from the organ 28. To facilitate the release of isolated cells, the agitation 10 members 14 are moved, for example, by shaking the digestion chamber 12, so as to gently agitate the organ 28.

The physiologically compatible medium exits the digestion chamber 12 through outlet 18 and conduit 41. The physiologically compatible medium can then be cooled. The physiologically compatible medium can be cooled by any suitable means, such as by flowing the physiologically compatible medium through a cooling device 44. The physiologically compatible medium can be cooled to a temperature between 5° C.-20° C. The cooling of the physiologically compatible medium can slow the activity of the protease and therefore, prevent damage to any of the isolated cells that have been released from the organ 28 and may be suspended in the physiologically compatible medium. However, the physiologically compatible medium can also bypass the cooling device 44 by manipulating valve 42 and flowing physiologically compatible medium through conduit 45 to valve 46.

Furthermore, after the physiologically compatible medium exits the digestion chamber 12, the user can monitor the physiologically compatible medium for the presence of isolated cells, such as at a sampling port 40. Once the user detects isolated cells, the physiologically compatible medium containing the isolated cells should not be re-circulated to the digestion chamber 12. The valve 46 can be used to direct the physiologically compatible medium containing isolated cells through a conduit 49 to a collector 48. Valve 46 can be used to direct the physiologically compatible medium that does not contain isolated cells through conduit 43 to be re-circulated by pump 32.

Additional physiologically compatible medium, such as in container 30, can be added to the system through conduit 31 and eventually to the digestion chamber 12 to facilitate the release of the cells from the organ 28. However, in another arrangement, the additional physiologically compatible medium may be added directly to the digestion chamber 12. The additional physiologically compatible medium can also be heated by the heating apparatus 36 prior to entering the digestion chamber 12; however, by manipulating valve 34 and valve 38, the additional physiologically compatible medium can bypass the heating apparatus 36.

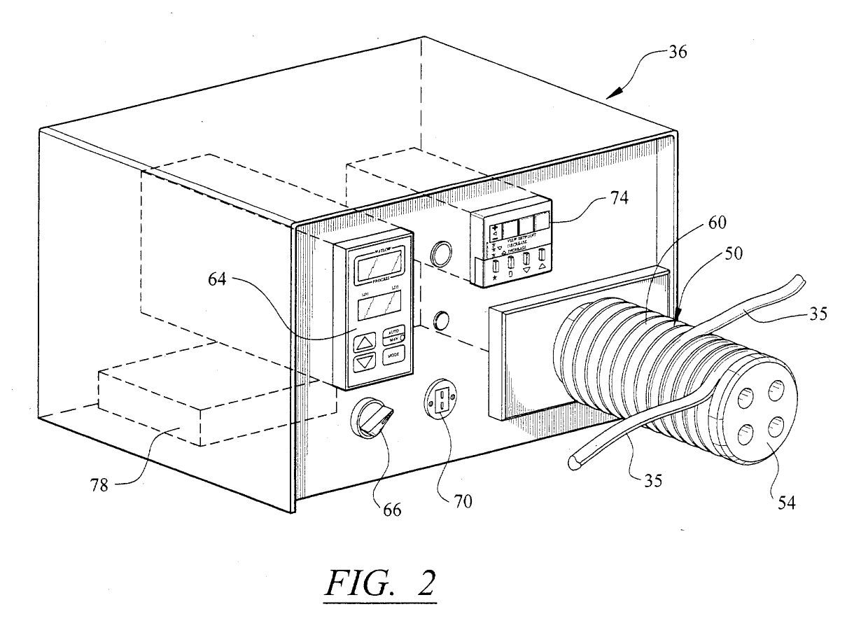

The conduit 35 leading to the heating apparatus 36 is typically flexible tubing. A heating apparatus 36 according to the invention is shown in FIG. 2. In the embodiment shown, the heating apparatus 36 comprises a heating surface 50 on a substantially cylindrical housing 54. Although the housing 54 is shown as substantially cylindrical, other shapes are possible. The heating surface 50 can be formed integrally with the housing 54, or can be a separate structure that is fixed to the surface of the housing 54.

The heating surface 50 can have any suitable shape. In the embodiment shown, the heating surface 50 is substantially cylindrical to facilitate the wrapping of flexible conduit over the heating surface 50 so as to contact the conduit with the heating surface 50. Other shapes are possible, including arcuate shapes. Flat heating surfaces are also possible in which the conduit can be coiled or otherwise secured, such as in a serpentine fashion, in order to facilitate contact between the coil and the heating surface.

Structure is preferably provided for removably retaining the flexible conduit in position against the heating surface 50. In the embodiment shown, the heating surface 50 includes grooves 60 formed therein. The grooves 60 are adapted to receive the conduit 35 when the conduit is wrapped about the housing 54. The grooves 60 assist in retaining the conduit in position in contact with the heating surface 50. Other structure for retaining the conduit in position on the heating surface 50 is possible. Such structure can include covers, clamps, ties, guides, straps, and the like. The conduit 35 for supplying physiologically compatible medium to the digestion chamber 12 can be wrapped about the heating apparatus 36 as shown in FIG. 2. The conduit 35 is wrapped about the housing 54 so as to contact the heating surface 50. More or fewer wraps of the conduit 35 about the heating surface 50 can be made to control the amount of heat imparted to the conduit and the medium that is flowing through the conduit. The temperature of the physiologically compatible medium exiting the conduit 35 and entering the digestion chamber 12 can thereby be approximately controlled.

The heating surface 50 is heated by suitable structure. One such suitable structure includes resistance heating by a resistive heat element, although the invention is not limited in this regard. It is also possible to heat the heating surface by means other than resistive heating. For example, a hot fluid can be caused to flow through suitable heat transfer apparatus so as to transfer heat to the heating surface 50.

The heating apparatus 36 can include suitable apparatus for controlling the heat transfer of the heating surface 50. A heater temperature controller 64 can be provided to regulate the heating of the heating surface 50, and thereby also of the medium flowing through the conduit 35 in contact with the heating surface 35. An ON-OFF switch 66 can be provided to control power to the heating apparatus 36. A thermocouple connector 70 can be used to connect a thermocouple for sensing the temperature of the medium. A chamber temperature controller 74 senses the temperature in cell extraction chamber and controls the temperature. Power relays 78 and other components can be provided.

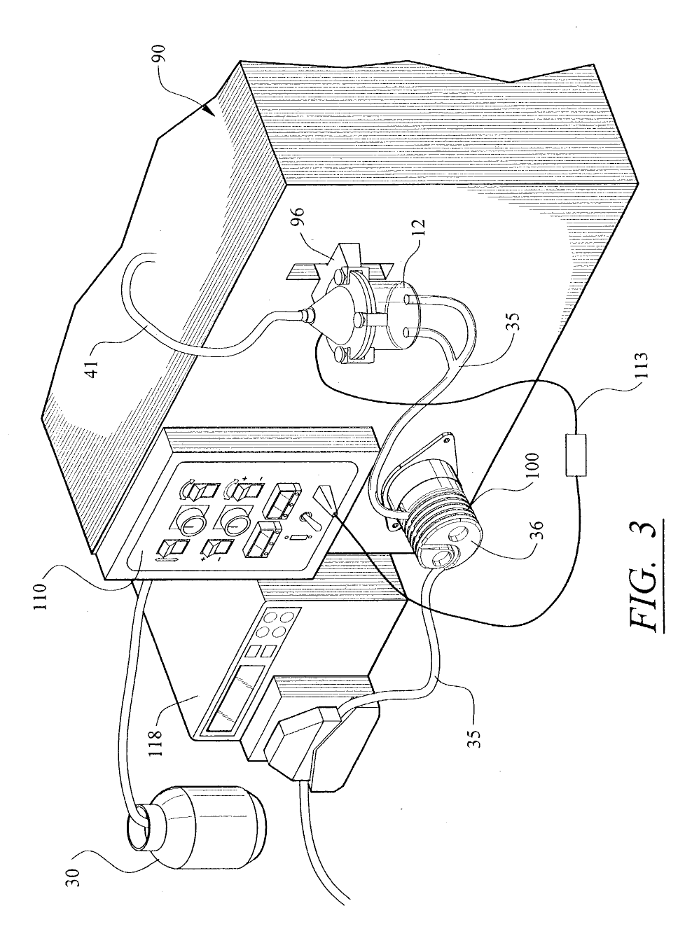

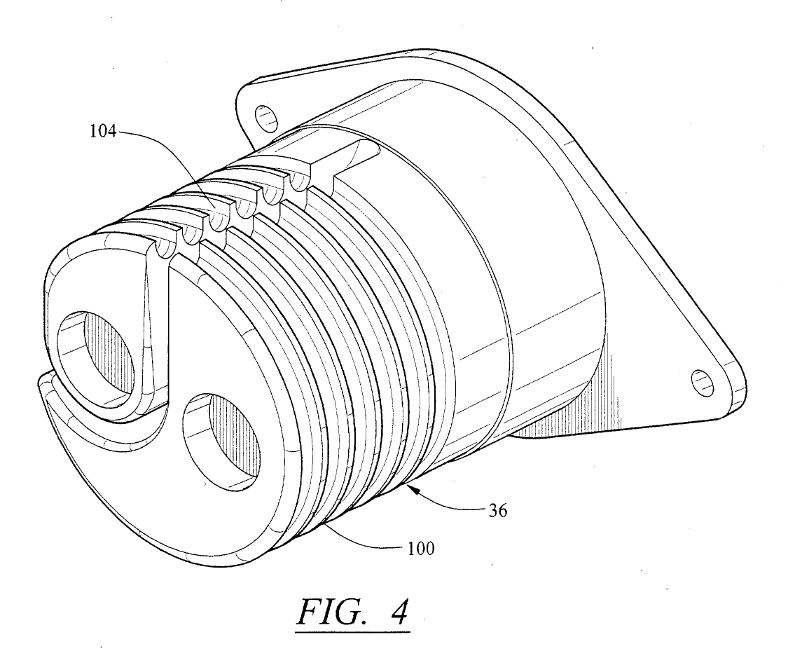

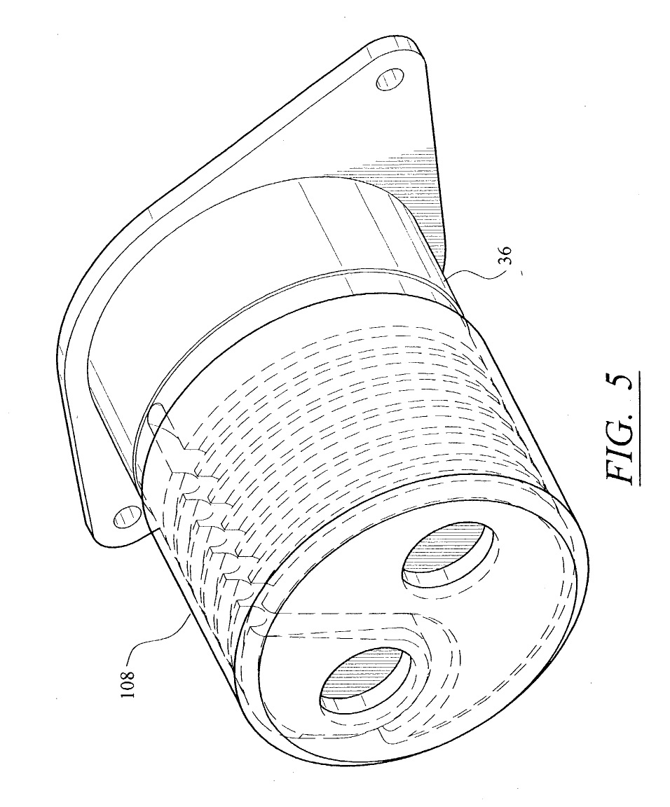

In FIG. 3 there is shown a cell extraction apparatus 90. The chamber 12 is mounted on a movable support 96 which moves the chamber 12 to provide agitation to facilitate cell extraction. The heating apparatus 36 is mounted on housing 98 of the apparatus 90. The flexible conduit 35 is contacted with the heating surface 100 of the heating apparatus 36 by wrapping the conduit 35 about the heating surface 100. The heating surface is substantially cylindrical to facilitate the wrapping of the conduit about the heating surface. The heating surface could alternatively be arcuate or provided on an arcuate or cylindrical housing. As shown in FIG. 4, the heating surface 100 comprises grooves 104 for retaining the conduit. As shown in FIG. 5, a cover 108 can be applied over the heating surface 100 in order to removably retain the conduit 35 in place. A control panel 110 is used to control the cell extraction apparatus 90. A pump 118 is used to pump the medium from a source container 30.

The heating apparatus 36 can be used in other processes besides extraction of cells from organs. The heating apparatus 36 has utility wherever fluids flowing through a flexible conduit must be heated. The invention permits the rapid adjustment of the temperature of the medium flowing through the conduit simply by contacting the conduit with the heating surface of the heating apparatus 36. Also, the invention provides for rapid process variation since the heating apparatus 36 does not come in contact with the physiologically compatible medium. The conduit need only be removed and replaced with a new conduit that is then contacted with the heating apparatus 36. No cleaning or sterilization of the heating apparatus 36 is necessary, and the invention provides for rapid process set-up and take down.

This invention can be embodied in other forms without departing from the spirit or essential attributes thereof. Accordingly, reference should be had to the following claims, rather than to the foregoing specification, as indicating the scope of the invention.

We claim:

1. A system for extracting cells from organs, comprising:
a digestion chamber for containing a physiologically compatible medium;
a conduit for supplying said physiologically compatible medium to said digestion chamber; and,
a heating apparatus having a heating surface and structure for removably securing a length of said conduit against said heating surface.

2. The system of claim 1, wherein said heating apparatus comprises a substantially cylindrical portion, said heating surface being provided at least in part on substantially cylindrical portion, whereby said conduit can be wrapped about said cylindrical portion and heated by said heating surface.

3. The system of claim 1, wherein said heating apparatus comprises a substantially cylindrical heating surface.

4. The system of claim 1, wherein said structure for removably securing said conduit against said heating surface comprises a cover adapted to fit over said heating surface and said conduit.

5. The system of claim 1, wherein said structure for removably securing said conduit against said heating surface comprises grooves in said heating surface for retaining said conduit.

6. The system of claim 1, said chamber having at least one inlet and at least one outlet, and a segregation means for retaining said organ and permitting cells to exit said outlet.

7. The system of claim 1, wherein said physiologically compatible medium comprises at least one protease.

8. The system of claim 1, further comprising a temperature contoller for controlling the heat transfer of the heating surface.

9. A method for extracting cells from organs, comprising the steps of:
providing a digestion chamber containing a physiologically compatible medium with at least one protease, said chamber having at least one inlet and at least one outlet, said chamber retaining said organ and permitting cells to exit said outlet;
providing a conduit for supplying said physiologically compatible medium to said digestion chamber;
providing a heating apparatus having a heating surface and structure for removably securing an adjustable length of said conduit against said heating surface of said heating apparatus;
placing a portion of said conduit against said heating surface; and,
flowing said physiologically compatible medium through said conduit to heat said physiologically compatible medium.

10. The method of claim 9, wherein said heating apparatus comprises a substantially cylindrical portion, said heating surface being provided at least in part on said substantially cylindrical portion, and said step of placing said conduit against said heating surface comprises wrapping said conduit about said substantially cylindrical portion.

11. The method of claim 9, further comprising the step of adjusting the temperature of physiologically compatible medium exiting said conduit, said adjusting step comprising adjusting the length of said conduit in contact with said heating surface.

12. The method of claim 5, wherein the temperature of said physiologically compatible medium leaving said heating apparatus is between 28° C. and 38° C.

13. The method of claim 5, wherein the temperature of said physiologically compatible medium leaving said heating apparatus is 37 C.

14. The method of claim 9, wherein said protease is collagenase.

15. A heating apparatus for heating a fluid flowing through a flexible conduit, comprising a heating surface and structure for removably securing an adjustable length of said conduit against said heating surface of said heating apparatus.

16. The heating apparatus of claim 15, wherein said heating surface is arcuate.

17. The heating apparatus of claim 15, wherein said heating surface is substantially cylindrical, such that said conduit can be wrapped about said cylindrical heating surface.

18. The heating apparatus of claim 15, wherein said heating apparatus comprises a substantially cylindrical housing, and said substantially cylindrical heating surface is provided on an outside surface of said housing.

19. The heating apparatus of claim 15 wherein said structure for removably securing said conduit to said heating apparatus comprises grooves for receiving said conduit.

20. The heating apparatus of claim 19, further comprising a cover adapted to fit over said heating surface and said conduit.
